(19) United States
(12) Patent Application Publication
(10) Pub. No.: US 2019/0000609 A1
(43) Pub. Date: Jan. 3, 2019

(54) CAPSULAR CLIP FOR CORRECTING ZONULAR WEAKNESS POST-CATARACT SURGERY

(71) Applicant: Baylor College of Medicine, Houston, TX (US)

(72) Inventor: Marshall Bowes Hamill, Houston, TX (US)

(73) Assignee: Baylor College of Medicine, Houston, TX (US)

(21) Appl. No.: 16/064,203

(22) PCT Filed: Dec. 15, 2016
(86) PCT No.: PCT/US16/66771

§ 371 Date: Jun. 20, 2018

Related U.S. Application Data
(60) Provisional application No. 62/270,270, filed on Dec. 21, 2015.

(51) Int. Cl. A61F 2/16

(52) U.S. Cl. CPC A61F 2/16; A61F 2220/0016; A61F 2002/16902

(57) ABSTRACT

When zonular attachments of a capsule holding an implanted lens weaken after cataract surgery, a capsular clip may be utilized to secure the capsular bag to the eye wall. The capsular clip may comprise an eyelet and a flat hook. The hook may be inserted either via a small surgeon created separation between the anterior capsule leaflet and the intraocular lens or via puncturing the capsular bag in a small area, and may be placed between the capsular bag and the implanted lens to secure the capsule in the appropriate position within the eye. Suture thread may be fed through the eyelet of the capsular clip to sew the capsular clip to the eye wall, thus securing the capsular clip and the capsule in place.

### CROSS-REFERENCE TO RELATED PATENT APPLICATIONS

This application claims the benefit of priority to U.S. Provisional Application No. 62/270,270 to Marshall Bowes Hamill filed Dec. 21, 2015 and entitled “Capsular Clip for Correcting Zonular Weakness Post-Cataract Surgery.”

### FIELD OF THE DISCLOSURE

The instant disclosure relates to a device and method for securing the capsular bag within the eye. More specifically, this disclosure relates correcting zonular weaknesses in a capsular bag after cataract surgery.

### BACKGROUND

Modern cataract surgery involves a making a small incision into the eye, opening the capsule of the cataractous lens, removing the cataract inside the capsule, and implanting an artificial plastic lens into the now empty capsular bag so as to provide focal power to the eye following removal of the cataract. The capsule itself is supported to the eye wall by many small filamentous attachments known as zonules extending 360° around the equator of the capsule. In most cases the zonular attachments are stable and sufficient to support the capsule and lens in the appropriate position within the eye for the life of the patient.

In some situations, however, these zonules may be weak due to previous trauma or surgery, may become weak because of trauma or surgery following a cataract operation, or may progressively weaken as the patient ages due to a congenital anomaly (such as pseudoexfoliation syndrome). The zonules may be so weak that they are no longer able to support the implant lens and capsular bag in its normal anatomical position. If the lens and capsular bag dislocate out of the proper position, the patient's vision may be decreased. If this situation occurs very early following cataract surgery, there are devices currently available that may be placed within the capsular bag so as to allow attachment of a suture and fixation to the eye wall. One example of such a device is shown in FIG. 1. Devices such as these are available as Ahmed Capsular Tension Segments (CTS). The CTS is a partial ring of clear PMMA covering approximately one quadrant with a hole for temporary or permanent fixation. A single iris retractor hooked into the hole can support the segment during surgery. Lacking a pointed tip, these broad, polished ring segments will not tear the capsulorhexis during surgery. Following surgery, they can either be removed or sutured to the sclera for permanent support.

However, if zonular weakness occurs late after the cataract operation (such as months or years later) the capsular bag becomes fibrotic and is no longer able to be opened so as to allow insertion of one of the currently available capsular support devices. In this situation, the current treatment is to remove the entire previously-implanted lens and sew a new implant within the eye, fixing the new implant lens to the eye wall with either sutures or glue at several locations. This requires fairly extensive surgery and manipulation of the intraocular tissues.

### SUMMARY

When zonular weakness occurs after cataract surgery, a capsular clip may be attached to the eye to secure the capsule in place. The clip may be inserted in a small and limited surgeon-created separation between the anterior capsule leaflet and the IOL anterior surface of the eye. The clip may alternatively or additionally be capable of being placed in a surgeon-created puncture of the capsule. The capsular clip may include a flat hook that may be used to attach to the capsular bag, such as via a surgeon-created small separation between the anterior capsular edge and the intraocular lens or utilizing a small puncture in the capsule, and the clip may also include an eyelet that may be used to sew the clip to the eye wall. The hook may be thin in thickness and narrow in width such that it only requires a small opening of the capsular bag. Such a small opening may be made even late after cataract surgery when the capsular bag is fibrotic. The use of this clip will allow the capsule housing the implanted lens to be reattached to the eye and supported without the need for exchange of the implant lens, creation of a large incision, or otherwise unnecessarily manipulating the intraocular tissues.

According to one embodiment, a capsular clip for attachment to an eye may comprise a flat hook having a shaft, a bend, and a tip for puncturing the capsular bag of a capsule holding an implanted lens; and an eyelet adapted for receipt of a suture thread therethrough for sewing the suture thread to an eye wall, and securing the capsule in place; wherein the thickness of the hook is less than the width of the hook at any point along the hook.

According to another embodiment, a method of attaching to an eye a capsular clip comprising an eyelet and a flat hook having a shaft, a bend, and a tip may include puncturing the capsular bag of a capsule holding an implanted lens using the tip of the hook; placing the hook such that the tip of the hook lies between the implanted lens and the membrane of the capsular bag, and such that the membrane of the capsular bag abuts the bend of the hook; and securing the capsular clip and the capsule in place by feeding suture thread through the eyelet of the capsular clip, and sewing the suture thread to the eye wall.

### BRIEF DESCRIPTION OF THE DRAWINGS

For a more complete understanding of the disclosed devices and methods, reference is now made to the following descriptions taken in conjunction with the accompanying drawings.

FIG. 1 is a perspective view of a prior art Ahmed Capsular Tension Segments (CTS).

FIG. 2 is a right side perspective view of a capsular clip according to some embodiments of the invention.

FIG. 3 is a right side elevational view of a capsular clip according to some embodiments of the invention.

FIG. 4 is a flow chart illustrating a method for attaching a capsular clip to the eye according to some embodiments of the disclosure.

FIG. 5 is an illustration of a capsular clip attached to the eye according to some embodiments of the disclosure.

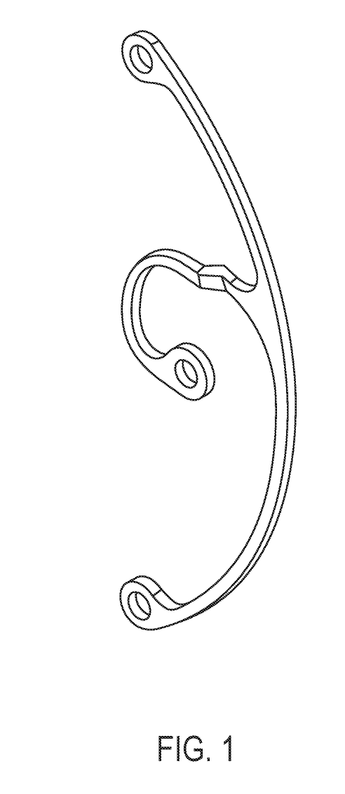

### DETAILED DESCRIPTION

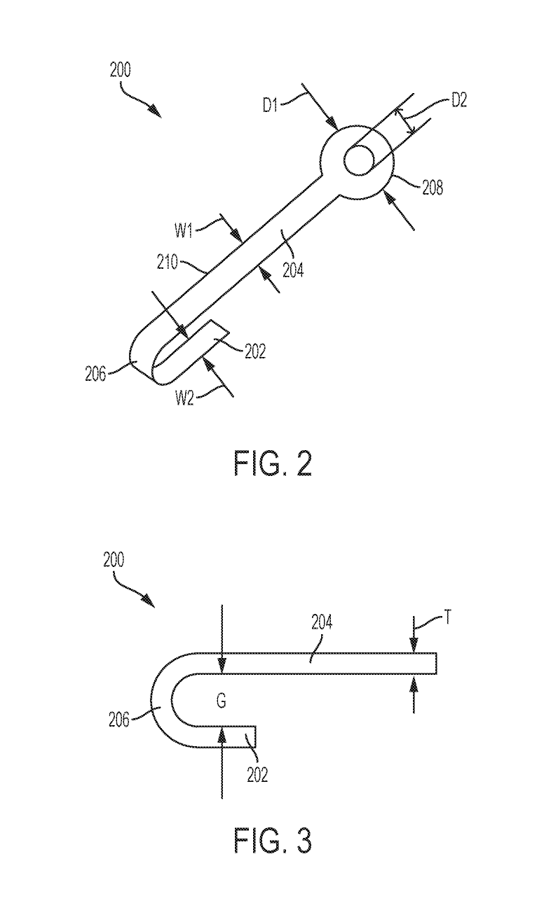

FIGS. 2-3 are illustrations showing a capsular clip, according to one embodiment of the disclosure. A capsular clip 200 may include an eyelet 208 that is contiguous with a hook 210. The hook 210 may comprise a shaft 204, a bend 206, and a tip 202. The capsular clip 200 may be made of a polymethyl methacrylate (PMMA) material, polypropylene, or other bio-compatible material. The capsular clip 200 may have thickness T; the outer edge of the eyelet 208 may have diameter D1; the eyelet 208 hole may have diameter D2; the hook shaft 204 may have width W1; the hook tip 202 may have width W2; and the distance between the hook shaft 204 and the hook tip 202 may be gap G.

In one embodiment, the hook 210 may be flat such that the thickness T of the hook 210 is less than the width of the hook at any point along the hook, such as W1 and W2. The hook 210 may be flat in shape, such that the cross-section at any location along the hook 210 is quadrilateral in shape. The distal portion of the hook 210 may be narrow and flat, such that it is able to fit between a capsule and an implant lens without significant dissection of a capsular bag and provide support to a fibrotic lens/capsule complex in the setting of previous cataract surgery and zonular weakness. In another embodiment, the distal portion of the hook 210 may be sharp to allow the clip 200 to be self-inserted into the eye.

Although dimensions are not critical to the use of the clip 200, some dimensions, which may be used as general guidelines, are provided in the follow description. In one embodiment, the width W1 of the hook shaft 204 may be equal to the width W2 of the hook tip 202. In an alternative embodiment, the width W1 of the hook shaft 204 may be greater than the width W2 of the hook tip 202. In one embodiment, the width W1 of the hook shaft 204 may be less than or equal to 0.5 mm, and the width W2 of the hook tip 202 may be less than or equal to 0.4 mm. The gap distance G between the hook shaft 204 and the hook tip 202 may be designed to match the thickness of the capsular bag membrane. For example, in one embodiment, this gap distance G may be 50 microns. The thickness T of the capsular clip 200 may be consistent throughout the entire clip. In one embodiment, the thickness T may be approximately 0.1 mm. In one embodiment, the eyelet 208 outer edge diameter D1 may be greater than the hook shaft 104 width W1. In one embodiment, the eyelet 208 outer edge diameter D1 may be approximately 1 mm. In one embodiment, the eyelet 208 hole diameter D2 may be less than or equal to 0.5 mm. Although certain dimensions are provided above as example sizes for various aspects of the hook 210, other sizes may be used for the hook 210 to accommodate a capsular bag.

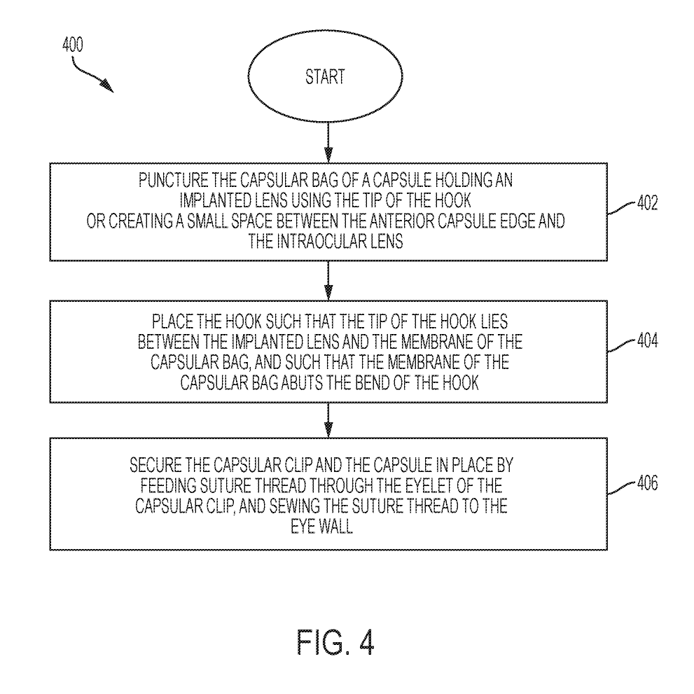

FIG. 4 is a flow chart illustrating a method for attaching a capsular clip to the eye, according to one embodiment of the disclosure. A method 400 may include attaching to an eye a capsular clip comprising an eyelet and a flat hook having a shaft, a bend, and a tip. The capsular clip used in the method in FIG. 4 may be the capsular clip described above with respect to FIGS. 2-3. The method 400 may start at block 402 with the surgeon either creating a small space between the anterior capsule edge and the intraocular lens or puncturing the capsular bag of a capsule holding an implanted lens using the tip of the hook. The tip of the hook may only create/require a small separation/opening. The tip of the hook may be capable of being inserted in a very small separation between the anterior capsule edge and the intraocular lens or puncturing a fibrous capsule. At block 404, the method may include placing the hook such that the tip of the hook lies between the implanted lens and the membrane of the capsular bag, and such that the membrane of the capsular bag abuts the bend of the hook. Once the hook is in such a position, the hook may be pulled to reposition the capsular bag. Once the hook has been pulled such that the capsule is positioned in its proper placement within the eye, such that the capsule does not decrease the patient's vision, the capsular clip may be sewn into place at the eyelet. At block 406, the method may include securing the capsular clip and the capsule in place by feeding suture thread through the eyelet of the capsular clip, and sewing the suture thread to the eye wall.

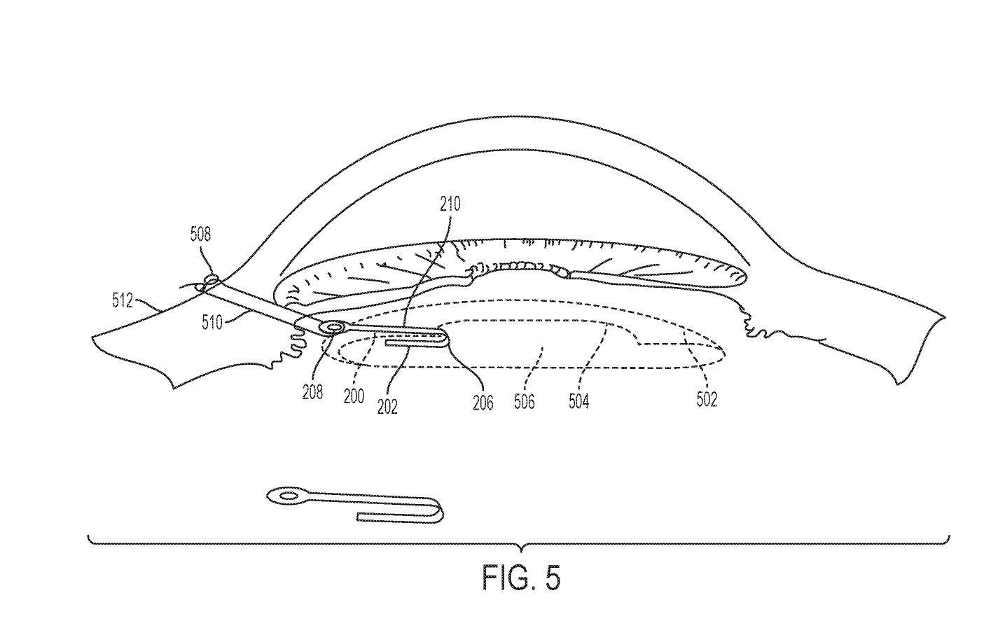

FIG. 5 is an illustration of a capsular clip attached to the eye, according to one embodiment of the disclosure. The capsular clip 200 may be placed within the capsular bag 502. The hook 210 of the capsular clip 200 may be placed such that the hook tip 202 lies between the implanted lens 506 and the membrane 504 of the capsular bag 502, and such that the membrane 504 of the capsular bag 502 abuts the bend 206 of the hook 210. Suture thread 510 may be fed through the eyelet 208 of the capsular clip 200 and sewn 508 to the eye wall 512 in order to hold the capsular bag 502 in place, such that it does not obstruct the patient's vision.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present invention, disclosure, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A capsular clip for attachment to an eye, comprising:
a flat hook comprising a shaft, a bend, and a tip configured to attach to a capsular bag of a capsule holding an implanted lens; and
an eyelet configured to receive a suture thread therethrough for sewing the suture thread to an anchor point within an eye to secure the capsule in place,
wherein the thickness of the hook is less than the width of the hook at any point along the hook.

2. The capsular clip of claim 1, wherein the tip is configured to attach by either creating a small separation between the anterior capsule leaflet and the intraocular lens or by puncturing the capsular bar.

3. The capsular clip of claim 1, wherein the capsular clip comprises polymethyl methacrylate (PMMA) material.

4. The capsular clip of claim 1, wherein the eyelet is configured to receive a suture and attach to an eye wall.

5. The capsular clip of claim 1, wherein the width of the hook shaft is less than or equal to 0.5 mm.

6. The capsular clip of claim 1, wherein the width of the hook tip is less than or equal to 0.4 mm.

7. The capsular clip of claim 1, wherein the thickness of the hook is less than or equal to 0.1 mm.

8. The capsular clip of claim 1, wherein the distance between the tip of the hook and the shaft of the hook is approximately 50 microns.

9. The capsular clip of claim 1, wherein the diameter of the eyelet hole is 0.5 mm.

10. A method of attaching to an eye a capsular clip comprising an eyelet and a flat hook comprising a shaft, a bend, and a tip, the method comprising the steps of:
attaching the capsular bag of a capsule holding an implanted lens using the tip of the hook;
placing the hook such that the tip of the hook lies between the implanted lens and the membrane of the capsular bag, and such that the membrane of the capsular bag abuts the bend of the hook; and
securing the capsular clip and the capsule in place by feeding suture thread through the eyelet of the capsular clip, and sewing the suture thread to the eye wall.

11. The method of claim 10, wherein the width of the hook shaft is less than or equal to 0.5 mm.

12. The method of claim 10, wherein the width of the hook tip is less than or equal to 0.4 mm.

13. The method of claim 10, wherein the thickness of the hook is less than or equal to 0.1 mm.

14. The method of claim 10, wherein the distance between the tip of the hook and the shaft of the hook is approximately 50 microns.

15. The method of claim 10, wherein the diameter of the eyelet hole is 0.5 mm.
